(19) United States
(12) Patent Application Publication
(10) Pub. No.: US 2024/0324677 A1
(43) Pub. Date: Oct. 3, 2024

(54) ATOMIZATION CORE, ATOMIZER AND ELECTRONIC ATOMIZATION APPARATUS

(71) Applicant: Hainan Moore Brothers Technology Co., Ltd., Hainan (CN)

(72) Inventors: Qijie SHEN, Hainan (CN); Zhenlong JIANG, Hainan (CN); Congwen XIAO, Hainan (CN); Hongxia LV, Hainan (CN); Hongliang LUO, Hainan (CN)

(21) Appl. No.: 18/735,661

(22) Filed: Jun. 6, 2024

Related U.S. Application Data
(63) Continuation of application No. PCT/CN2021/136172, filed on Dec. 7, 2021.

(51) Int. Cl. A24F 40/46; A24F 40/10; A24F 40/44; A61M 11/04; A61M 15/06; H05B 3/22

(52) U.S. Cl. CPC A24F 40/46; A24F 40/10; A24F 40/44; A61M 11/042; A61M 15/06; H05B 3/22; A61M 2205/0211; A61M 2205/0233

(57) ABSTRACT

An atomization core includes: a substrate having an atomization surface and through holes extending to the atomization surface, the substrate guiding an aerosol-forming material to the atomization surface; a heating element arranged on the atomization surface for heating and atomizing the aerosol-forming material to form aerosols; and conductor leads arranged in the through holes and fixed to the substrate to form an integral structure, a first end of each conductor lead of the conductor leads being electrically connected to the heating element, and a second end of each conductor lead of the conductor leads connecting a power supply assembly.

### CROSS-REFERENCE TO PRIOR APPLICATION

This application is a continuation of International Patent Application No. PCT/CN2021/136172, filed on Dec. 7, 2021. The entire disclosure is hereby incorporated by reference herein.

### FIELD

This application relates to the technical field of an atomizer, and particularly relates to an atomization core, an atomizer and an electronic atomization apparatus.

### BACKGROUND

In the related art, an electronic atomization apparatus mainly consists of an atomizer and a power supply assembly. An atomization core in the atomizer is a core component, and the atomization core mainly includes a ceramic atomization core and a liquid guide cotton atomization core. The traditional ceramic atomization core is obtained by forming a layer of heating film resistor on the surface of the ceramic substrate through screen printing and sintering and a group of conductor leads connected to a power supply through sintering attaching. However, the contact stability between conductor leads and a substrate of the ceramic atomization core is poor, and damage may easily occur.

### SUMMARY

In an embodiment, the present invention provides an atomization core, comprising: a substrate having an atomization surface and through holes extending to the atomization surface, the substrate being configured to guide an aerosol-forming material to the atomization surface; a heating element arranged on the atomization surface and configured to heat and atomize the aerosol-forming material to form aerosols; and conductor leads arranged in the through holes and fixed to the substrate to form an integral structure, a first end of each conductor lead of the conductor leads being electrically connected to the heating element, and a second end of each conductor lead of the conductor leads being configured to connect a power supply assembly.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an electronic atomization apparatus provided by this application;

FIG. 2 is a schematic structural diagram of an atomizer provided by this application;

FIG. 3 is a schematic structural diagram of an atomization core in an embodiment provided by this application.

FIG. 4 is a front-view schematic structural diagram of the atomization core provided in FIG. 3;

FIG. 5 is a bottom-view schematic structural diagram of the atomization core provided in FIG. 3;

FIG. 6 is a cross-sectional view of a first embodiment of the atomization core provided in FIG. 5 in Direction A-A;

FIG. 7 is a cross-sectional view of a second embodiment of the atomization core provided in FIG. 5 in Direction A-A;

FIG. 8 is a cross-sectional view of a third embodiment of the atomization core provided in FIG. 5 in Direction A-A;

FIG. 9 is a cross-sectional view of a fourth embodiment of the atomization core provided in FIG. 5 in Direction A-A;

FIG. 10 is a schematic structural diagram of an atomization core in another embodiment provided by this application;

FIG. 11 is a lateral cross-sectional view of connection between conductor leads and a substrate in an embodiment provided by this application;

FIG. 12 is a cross-sectional view of a structure of a first embodiment of the conductor leads provided by this application;

FIG. 13 is a cross-sectional view of a structure of a second embodiment of the conductor leads provided by this application;

FIG. 14 is a cross-sectional view of a structure of a third embodiment of the conductor leads provided by this application; and

FIG. 15 is a cross-sectional view of a connection structure of the substrate and the conductor leads provided by this application.

### DETAILED DESCRIPTION

In an embodiment, the present invention provides an atomization core, an atomizer and an electronic atomization apparatus to solve the problems that the contact stability between conductor leads and a substrate of a ceramic atomization core is poor, and damage may easily occur in the prior art.

In an embodiment, the present invention provides an atomization core that includes a substrate, a heating element and conductor leads. The substrate has an atomization surface and through holes extending to the atomization surface. The substrate is configured to guide an aerosol-forming material to the atomization surface. The heating element is arranged on the atomization surface and is configured to heat and atomize the aerosol-forming material to form aerosols. The conductor leads are arranged in the through holes and fixed to the substrate to form an integral structure. A first end of each of the conductor leads is electrically connected to the heating element, and a second end of each of the conductor leads is configured to connect a power supply assembly.

The conductor leads have protrusions on the side walls, the through holes have depressions on the side walls, and the protrusions are embedded in the depressions.

The conductor leads are solid conductors, or the conductor leads have pores.

The solid parts of the conductor leads account for more than 50% of the volume of the through holes.

The substrate has a first surface and a second surface in opposite arrangement, and the first surface is the atomization surface. The through holes extend from the first surface to the second surface.

The through holes are straight-through holes perpendicular to the first surface.

The atomization core further includes electrodes and bonding pads. The electrodes are arranged on the first surface and is electrically connected to the heating element, and the bonding pads are arranged on the second surface and are configured to be connected to the power supply assembly.

The first end of each of the conductor leads is electrically connected to the electrode, and the second end of each of the conductor leads is electrically connected to the bonding pad.

The lead diameter of each of the conductor leads ranges from 0.1 mm to 1 mm, and/or, a material used by the conductor leads is one or more of Ag, Cu and Au.

The substrate is a porous substrate. The porosity of the substrate ranges from 30% to 80%, and/or the pore diameter of the pores of the substrate ranges from 10 um to 200 um.

The conductor leads are prepared by a method of filling conductive paste into the through holes and then performing sintering.

The substrate has a first surface and a second surface in opposite arrangement, and side surfaces connected to the first surface and the second surface, the first surface is the atomization surface, and the through holes extend from the first surface to the side surfaces.

To solve the foregoing technical problems, a second technical solution provided by this application is as follows: An atomizer is provided and includes a housing and an atomization core. The housing has an accommodating cavity. The atomization core is arranged in the accommodating cavity, and is matched with the housing to form a liquid storage cavity. The atomization core is configured to heat and atomize an aerosol-forming material from the liquid storage cavity when powered on to form aerosols, and the atomization core is any one of the atomization core described above.

To solve the foregoing technical problems, a third technical solution provided by this application is as follows: An electronic atomization apparatus is provided and includes an atomizer and a power supply assembly. The atomizer is any one of the atomizer described above. The power supply assembly is electrically connected to conductor leads of the atomizer and is configured to supply power to the atomizer.

This application has the following beneficial effects: This application differs from the prior art in that the atomization core of this application includes a substrate, a heating element and conductor leads. The substrate has an atomization surface and through holes extending to the atomization surface. The substrate is configured to guide an aerosol-forming material to the atomization surface. The heating element is arranged on the atomization surface and is configured to heat and atomize the aerosol-forming material to form aerosols. The conductor leads are arranged in the through holes and fixed to the substrate to form an integral structure. A first end of each of the conductor leads is electrically connected to the heating element, and a second end of each of the conductor leads is configured to connect a power supply assembly. In this application, the conductor leads are arranged in the substrate to form an integral structure with the substrate, so that the connection direction of the heating element and a power supply is changed, the leads are prevented from shielding the atomization surface, the problems that the contact stability between the conductor leads and the substrate of the ceramic atomization core is poor, and damage may easily occur are solved, and the atomization conversion efficiency is improved to a greatest extent.

The technical solutions in embodiments of this application are clearly and completely described in the following with reference to the accompanying drawings in the embodiments of this application. Apparently, the described embodiments are merely some rather than all of the embodiments of this application. All other embodiments obtained by a person of ordinary skill in the art based on the embodiments of this application without creative efforts shall fall within the protection scope of this application.

The terms “first” and “second” in this application are merely intended for a purpose of description, and shall not be understood as indicating or implying relative significance or implicitly indicating the number of indicated technical features. Therefore, features defining “first” or “second” may explicitly or implicitly include at least one of the features. All directional indications (for example, up, down, left, right, front, back . . . ) in the embodiments of this application are only used for explaining relative position relationships, movement situations, or the like between the various components in a specific posture (as shown in the accompanying drawings). If the specific posture changes, the directional indications change accordingly. In addition, the terms “include”, “have”, and any variant thereof are intended to cover a non-exclusive inclusion. For example, a process, method, system, product, or device that includes a series of steps or units is not limited to the listed steps or units; and instead, further optionally includes a step or unit that is not listed, or further optionally includes another step or unit that is intrinsic to the process, method, product, or device. Embodiment mentioned in the specification means that particular features, structures, or characteristics described with reference to the embodiment may be included in at least one embodiment of this application. The term appearing at different positions of this specification may not refer to the same embodiment or an independent or alternative embodiment that is mutually exclusive with another embodiment. A person skilled in the art explicitly or implicitly understands that the embodiments described in the specification may be combined with other embodiments.

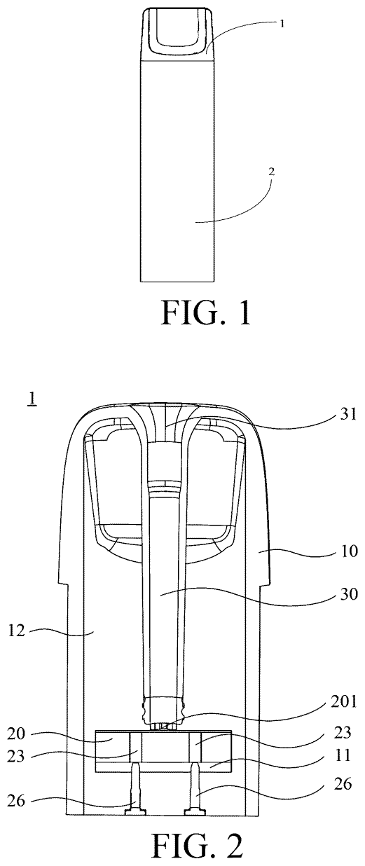

Referring to FIG. 1, FIG. 1 is a schematic structural diagram of an electronic atomization apparatus provided by this application.

The electronic atomization apparatus includes an atomizer 1 and a power supply assembly 2. The power supply assembly 2 is connected to the atomizer 1, and is configured to supply power to the atomizer 1. The electronic atomization apparatus may be configured to atomize a liquid substrate. The atomizer 1 is configured to store a liquid aerosol-forming material and atomize the aerosol-forming material to form aerosols capable of being inhaled by a user. The liquid aerosol-forming material may be a liquid substrate such as a medicinal liquid and a plant grass leaf type aerosol-forming material. The atomizer I may be specifically used in different fields, such as medical treatment, beauty treatment and recreational inhalation. The power supply assembly 2 includes a battery, an airflow sensor, and a controller. The battery is configured to supply power to the atomizer 1 and control the heating power, heating duration, etc. of an atomization core 20 so that the atomizer 1 may atomize an aerosol-forming material to form aerosols. The airflow sensor is configured to detect an airflow change in the electronic atomization apparatus, and the controller starts the electronic atomization apparatus according to the airflow change detected by the airflow sensor. The atomizer 1 and the power supply assembly 2 may be arranged integrally or may be detachably connected, which is designed according to specific requirements.

Referring to FIG. 2, FIG. 2 is a schematic structural diagram of an atomizer provided by this application.

The atomizer 1 includes a housing 10 and an atomization core 20. The housing 10 is provided with an accommodating cavity 11. The atomization core 20 and the housing 10 may be nondetachably connected in an integral arrangement manner, and may also be detachably connected. In this embodiment, the atomization core 20 and the housing 10 are detachably connected, and the atomization core 20 is directly connected to the housing 10, so that the atomization core 20 and the housing 10 may be detachably connected without introducing an additional conduit, the volume of the atomizer 1 is reduced, and the use is more convenient. It can be understood that the atomizer 1 of this application is a portable atomizer. The atomization core 20 is arranged in the accommodating cavity 11, is matched with the housing 10 to form a liquid storage cavity 12, and is configured to store an aerosol-forming material. The atomization core 20 may be used in different fields, such as medicine atomization and oil flower and grass liquid atomization, and is configured to heat and atomize the aerosol-forming material from the liquid storage cavity 12 when powered on to form aerosols. The atomizer 1 may further include an installing seat, and the installing seat is configured to install the atomization core 20.

Specifically, protrusions are formed on the outer wall surface of the atomization core 20, chutes are formed on the outer wall surface of the housing 10, and limiting blocks are arranged in the chutes. The protrusions on the atomization core 20 are aligned with and inserted into the chutes of the housing 10, the atomization core 20 or the housing 10 is rotated so that the protrusions are limited by the limiting blocks in the chutes, the fixation of the atomization core 20 to the housing 10 is realized, and the detachable connection between the atomization core 20 and the housing 10 is realized. It can be understood that protrusions may also be formed on the outer wall surface of the housing 10, chutes may be formed on the outer wall surface of the atomization core 20, limiting blocks may be arranged in the chutes, and the detachable connection between the atomization core 20 and the housing 10 is realized. The detachable connection between the atomization core 20 and the housing 10 may also be realized by using a magnetic suction manner. The specific implementation is not limited as long as the detachable connection between the atomization core 20 and the housing 10 is realized.

In an embodiment, the atomization surface of the atomization core 20 faces an upward direction, and the atomization amount may be improved. When the atomization surface faces the upward direction, a pin of the atomization core 20 may be arranged in any one position of the atomization core 20. In this embodiment, the pin is arranged downwards, and the atomizer 1 may be conveniently and automatically assembled. An inhaling passage 30 is arranged on one side of the atomization core 20 far away from the power supply assembly 2, and the inhaling passage 30 communicates with an atomization cavity 201. An inhaling opening 31 at one side of the inhaling passage 30 far away from the power supply assembly 2 communicates with the atmosphere, so that the aerosols in the atomization cavity 201 may flow out through the inhaling passage 30 and may be provided for a user to be inhaled from the inhaling opening 31.

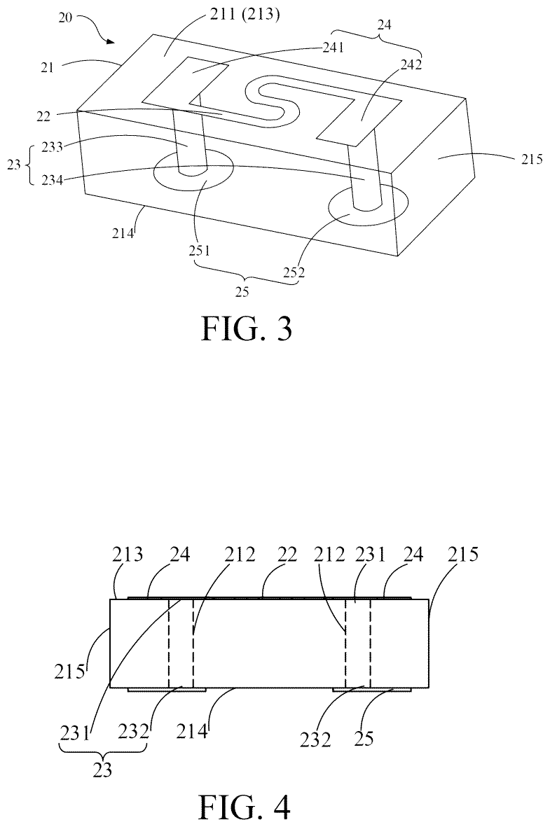

Referring to FIG. 3 and FIG. 4, FIG. 3 is a schematic structural diagram of an atomization core in an embodiment provided by this application, and FIG. 4 is a front-view schematic structural diagram of the atomization core provided in FIG. 3.

In an embodiment, the atomization core 20 includes a substrate 21, a heating element 22 and conductor leads 23.

Specifically, the substrate 21 may be a porous substrate or a perforated compact substrate. The porous substrate concretely may be a porous ceramic substrate, and the perforated compact substrate may be a perforated glass substrate or a compact ceramic substrate, etc. The substrate 21 in this embodiment is made of porous ceramic. The porous ceramic material is generally a ceramic material obtained by treating components such as an aggregate, a bonding agent and a pore former through high-temperature sintering. A great number of pore structures which communicate with each other and communicate with the surface of the material are formed inside the porous ceramic material. Due to its good performance such as high porosity, stable chemical properties, large specific surface area, small volume density, low heat conductivity, high-temperature resistance and corrosion resistance, the porous ceramic material has wide application in the fields of metallurgy, biology, energy source, environment protection, etc.

The substrate 21 may be in a flat plate shape or a step shape, and it is not specifically limited by this application. The substrate 21 has a first surface 213 and a second surface 214, the first surface 213 is the surface of the substrate 21 at one side facing the liquid storage cavity 12, and the second surface 214 is the surface of the substrate 21 at one side facing away from the first surface 213. Both the first surface 213 and the second surface 214 may be flat planes, the first surface 213 and the second surface 214 may also be irregular surfaces such as curve surfaces, and they are not specifically limited by this application. For example, a groove is formed in one side of the first surface 213 of the substrate 21, and the surface of the groove also belongs to the first surface 213.

The substrate 21 has an atomization surface 211 and through holes 212 extending to the atomization surface 211, and is configured to guide an aerosol-forming material to the atomization surface 211. In this embodiment, the porosity of the substrate 21 ranges from 30% to 80%, and/or the pore diameter of the pores of the substrate 21 ranges from 10 um to 200 um. It can be understood that the higher the porosity of the substrate 21 is, the higher the liquid guide speed is. Meanwhile, the pore diameter of the pores of the substrate 21 is associated with protrusions of the conductor leads 23. Due to this range, the matching rate of the conductor leads 23 and the substrate 21 may be the highest. Meanwhile, the conduction of the conductor leads 23 and the flow guide on the aerosol-forming material by the substrate 21 may be convenient. In this embodiment, the hole diameter of the through holes 212 ranges from 0.1 mm to 1 mm. In other embodiments, the hole diameter of the through holes 212 and the porosity of the substrate 21 may be set according to requirements, and it is not limited in this application.

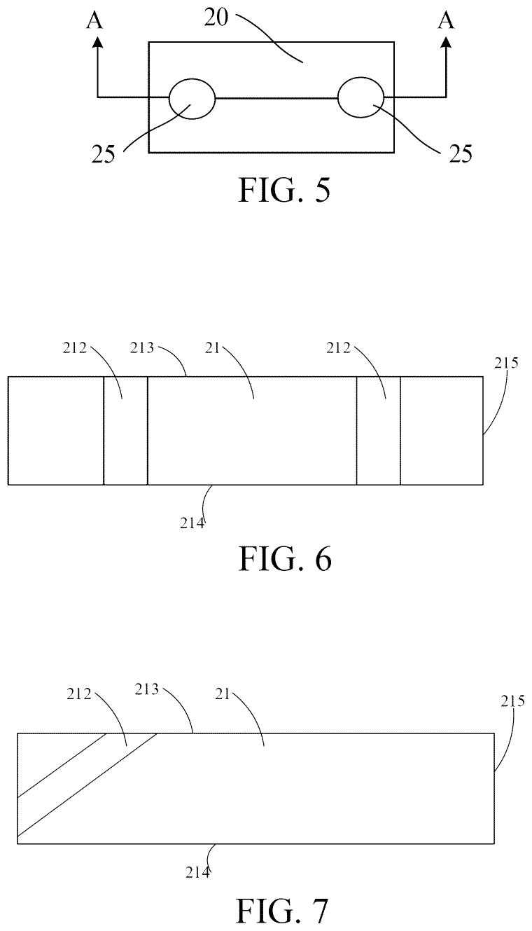

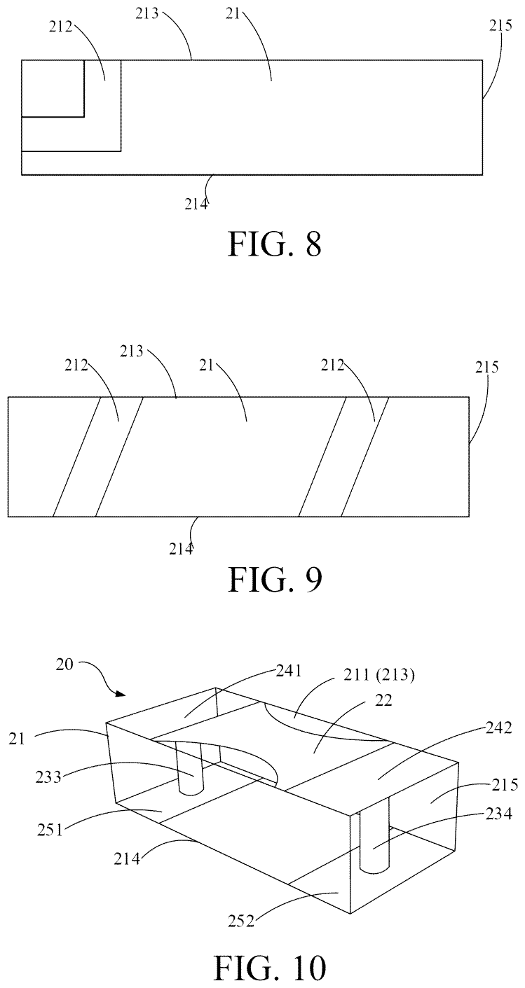

Referring to FIG. 5 to FIG. 8, FIG. 5 is a bottom-view schematic structural diagram of the atomization core provided in FIG. 3. FIG. 6 is a cross-sectional view of a first embodiment of the atomization core provided in FIG. 5 in Direction A-A. FIG. 7 is a cross-sectional view of a second embodiment of the atomization core provided in FIG. 5 in Direction A-A. FIG. 8 is a cross-sectional view of a third embodiment of the atomization core provided in FIG. 5 in Direction A-A. FIG. 9 is a cross-sectional view of a fourth embodiment of the atomization core provided in FIG. 5 in Direction A-A.

In an embodiment, the substrate 21 has a first surface 213, a second surface 214 and a side surface 215. The second surface 214 and the first surface 213 are arranged facing away from each other, and the side surface 215 is connected to the first surface 213 and the second surface 214. Generally, the first surface 213 may be configured to be in contact with the aerosol-forming material communicating with the liquid storage cavity 12, the second surface 214 may be configured to be in contact with gas. The contact with gas mentioned herein may refer to that the second surface 214 is in contact with external air, in contact with air in the atomization cavity 201, or in contact with the air in the inhaling passage 30, etc.

In this embodiment, the aerosol-forming material located at one side of the second surface 214 of the substrate 21 penetrates to the side where the first surface 213 of the substrate 21 is located through a great number of pore structures which communicate with each other and communicate with the surface of the material inside the substrate 21, the heating element 22 is arranged on the first surface 213 to atomize the aerosol-forming material penetrating to the first surface 213. Pore structures also communicate with the side surface 215. Therefore, the side surface 215 may also be used for liquid guide or ventilation.

As shown in FIG. 3 and FIG. 4, in the first embodiment, the substrate 21 has the first surface 213 and the second surface 214 in opposite arrangement, and the first surface 213 is the atomization surface 211. The through holes 212 extend from the first surface 213 to the second surface 214, and liquid may be conveniently sucked from the side surface 215 for forming aerosol. Meanwhile, liquid may be sucked from the second surface 214 for forming aerosol.

In the first embodiment, the through holes 212 are straight-through holes perpendicular to the first surface 213, so that the through holes may be conveniently prepared. If the through holes 212 are straight-through holes perpendicular to the first surface 213, meanwhile, a raw material of the substrate may be perforated to form multiple through holesin one step, and may be cut into a plurality of substrates 21. Meanwhile, mold preparation may also be convenient, and the efficiency is high.

In the second embodiment, the substrate 21 also has the first surface 213 and the second surface 214 in opposite arrangement, and the side surface 215 connected to the first surface 213 and the second surface 214. The first surface 213 is the atomization surface 211, and the through holes 212 may extend from the first surface 213 to the side surface 215. Specifically, as shown in FIG. 7, the through hole 212 may be an inclined hole structure extending to the side surface 215. That is, the through hole 212 is a straight hole facing the side surface 215.

As shown in FIG. 8, in the third embodiment, the through hole 212 may also be a cornered through hole connected to the side surface 215 and the first surface 213. When the through hole 212 is perforated through extending from the first surface 213 to the side surface 215, mold stripping is difficult, meanwhile, the mold cannot perforated to form multiple through holes, and the perforation efficiency is low. However, the through hole 212 is of a structure extending from the first surface 213 to the side surface 215, liquid suction from the second surface 214 may be realized, and such a structure is suitable for a downward-atomization atomizer. The structure may be set according to requirements in practical use, and it is not limited in this application.

As shown in FIG. 9, in the fourth embodiment, the through hole 212 may further be a straight-through hole/non-straight-through hole forming a certain oblique angle with the first surface 213 or the side surface 215. When the through hole 212 is the straight-through hole/non-straight-through hole forming a certain oblique angle, identically, mold stripping is difficult, meanwhile, the mold cannot perforated to form multiple through holes, and the perforation efficiency is low. However, liquid suction from the second surface 214 may be realized, and such a structure is suitable for a downward-atomization atomizer. The structure may be specifically set according to requirements. It can be understood that the quantity of the through hole(s) 212 may be one or more, and the extending direction may be parallel or non-parallel as long as they are matched with the quantity of the conductor leads 23, and they are not limited in this application.

As shown in FIG. 3 and FIG. 4, the heating element 22 is arranged on the atomization surface 211, and the heating element 22 is configured to atomize an aerosol-forming material guided out through the substrate 21. The atomization surface 211 absorbs heat of the heating element 22, and is thus configured to heat and atomize an aerosol-forming material when powered on to form aerosols. In this embodiment, the heating element 22 uses a metal heating film which has a good heat conduction effect. In other embodiments, the heating element 22 may further be at least one of a heating coating, a heating line, a heating sheet or a heating net, and it is not limited in this application.

In this embodiment, a porous ceramic material is used for making the substrate 21, the aerosol-forming material located at one side of the substrate 21 penetrates to the other side of the substrate 21 through a great number which communicate with each other and communicate with the material surface inside the porous ceramic material, and is additionally in contact with the heating element 22 arranged on one side surface of the substrate 21, so that the aerosol-forming material is atomized into the aerosols.

As shown in FIG. 3, in an embodiment, the heating element 22 is in an S shape, the heating element 22 may be of an integrally formed structure or a detachable structure, and it may be set according to requirements. In this embodiment, the thickness of the metal heating film used by the heating element 22 ranges from 50 um to 120 um, the heating efficiency is high, the heat conductivity is high, and the atomization efficiency of the aerosol-forming material can be improved. In other embodiments, the heating element 22 may further be in a rectangular shape, an oval shape or a round shape, etc., additionally, the thickness, size, quantity, etc. of the heating element 22 may be set according to requirements, and they are not limited in this application.

As shown in FIG. 3, in an embodiment, the atomization core 20 further includes electrodes 24 and bonding pads 25. The electrodes 24 are arranged on the first surface 213 and are electrically connected to the heating element 22. The bonding pads 25 are arranged on the second surface 214 and are configured to be connected to the power supply assembly 2.

Specifically, the electrodes 24 include a first electrode 241 and a second electrode 242, and the first electrode 241 and the second electrode 242 are arranged at interval and are both connected to the heating element 22. The heating element 22 is configured to atomize the aerosol-forming material guided out through the substrate 21. Specifically, the heating element 22 may further be at least one of a heating coating, a heating line, a heating sheet or a heating net, and the heating element 22 is electrically connected to the power supply assembly 2 through the electrodes 24. The first electrode 241 and the second electrode 242 may be arranged on a part of region on the atomization surface 211, and may extend to the edge of the atomization surface 211, and it is not limited in this application.

Specifically, as shown in FIG. 3, the quantity of the bonding pad(s) 25 may be one or more, the bonding pads may be set to be in a shape of a cylinder or a cuboid, and may be set specifically according to requirements as long as they are matched with the quantity of the conductor leads 23. One end of each conductor lead 23 is connected to the electrode 24, and the other end of each conductor lead is connected to the bonding pad 25. In this embodiment, two bonding pads 25 are used and include a first bonding pad 251 and a second bonding pad 252, and the first bonding pad 251 and the second bonding pad 252 are arranged at interval. The first bonding pad 251 and the second bonding pad 252 are respectively connected to the two conductor leads 23, and the conductor leads 23 vertically penetrate through the electrodes 24 and the bonding pads 25. Specifically, the conductor leads 23 includes a first conductor lead 233 and a second conductor lead 234, the first conductor lead 233 vertically penetrates through the first electrode 241 and the first bonding pad 251, the second conductor lead 234 vertically penetrates through the second electrode 242 and the second bonding pad 252, and the two ends of the first conductor lead 233 and the second conductor lead 234 are respectively and electrically connected to the first electrode 241/the second electrode 242 and the first electrode bonding pad 251/the second bonding pad 252. In other embodiments, the bonding pads 25 may further be arranged on the side surface 215, and the bending of the conductor leads 23 and the liquid suction shield of the second surface 214 may be avoided by adopting this arrangement manner. Therefore, the specific position of the bonding pads 25 may be set according to specific requirements, and it is not limited in this application.

Referring to FIG. 10, FIG. 10 is a schematic structural diagram of an atomization core in another embodiment provided by this application.

As shown in FIG. 10, in another embodiment, the heating element 22 is in a waist shape with two wide ends and the gradually decreased middle, the two ends of the heating element 22 are respectively connected to the first electrode 241 and the second electrode 242, and the first electrode 241 and the second electrode 242 are arranged at interval, and are both connected to the two ends of the waist-shaped heating element 22. Additionally, the first bonding pad 251 and the second bonding pad 252 are arranged on the second surface 214 of the substrate 21, and the first bonding pad 251 and the second bonding pad 252 are arranged at interval. The first conductor lead 233 is respectively connected to the first electrode 241 and the first bonding pad 251, and the second conductor lead 234 is respectively connected to the second electrode 242 and the second bonding pad 252. In this embodiment, the thickness of the metal heating film used by the waist-shaped heating element 22 ranges from 10 um to 50 um, the heating efficiency is high, the heat conductivity is high, and the atomization efficiency of the aerosol-forming material can be improved.

Optionally, the first electrode 241 and the first bonding pad 251 may be the electrode and the bonding pad with the same size of the laminated projection, and may further be the electrode and the bonding pad with different sizes. In some embodiments, the same structure arrangement with the laminated projection is adopted, and these two regions may be printed through the same printing screen, so the preparation is convenient.

As shown in FIG. 3, FIG. 4 and FIG. 10, in some embodiments, the conductor leads 23 are arranged in the through holes 212. Each of the conductor leads 23 includes a first end 231 and a second end 232, the first end 231 is electrically connected to the electrode 24, and the second end 232 is configured to connect the power supply assembly 2. Specifically, the first end 231 of each of the conductor leads 23 is electrically connected to the electrode 24, and the second end 232 is electrically connected to the bonding pad 25.

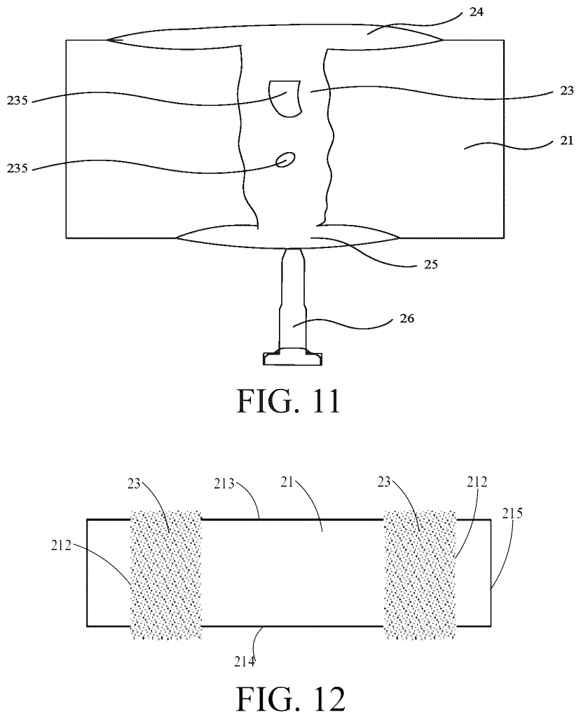

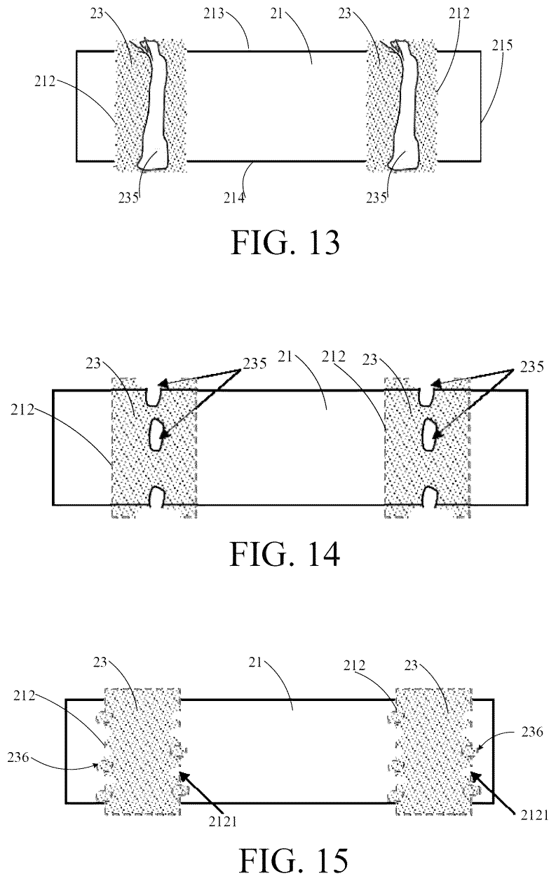

Referring to FIG. 11 to FIG. 15, FIG. 11 is a lateral cross-sectional view of connection between a conductor lead and a substrate in an embodiment provided by this application. FIG. 12 is a cross-sectional view of a structure of the first embodiment of the conductor leads provided by this application. FIG. 13 is a cross-sectional view of a structure of the second embodiment of the conductor leads provided by this application. FIG. 14 is a cross-sectional view of a structure of the third embodiment of the conductor leads provided by this application. FIG. 15 is a cross-sectional view of a connection structure of the substrate and the conductor leads provided by this application.

As shown in FIG. 11 to FIG. 15, in an embodiment, the conductor leads 23, the electrodes 24 and the bonding pads 25 are all arranged in the through holes 212, and are all prepared by a method of filling conductive paste into the through holes 212 and then performing sintering. Optionally, the lead diameter of each of the conductor leads 23 ranges from 0.1 mm to 1 mm. A material used by the conductor leads 23 is one or more of Ag, Cu and Au, and may be specifically designed according to requirements, and it is not limited in this application.

Specifically, in this embodiment, a metal or alloy material with the highest conductibility, such as one or a combination of more of Ag, Cu, Au, etc. is used, paste form metal of the metal or the alloy material is screen-printed and filled into the through holes 212 to be matched and co-sintered with the porous structure ceramic substrate 21 to form an integral structure of the conductor leads 23, the electrodes 24, the bonding pads 25 and the substrate 21. The integral structure may be a nondetachable structure. Specifically, the integral structure is not a structure which is perforated for inserting leads, and is also not a structure with the clamped substrate 21 and conductor leads 23.

Meanwhile, the integral structure is different from an integrally formed structure. It is an indivisible and nondetachable structure formed by the substrate 21 and the paste form of the metal or alloy material through co-sintering.

The conductor leads 23 after matching and co-sintering have the following characteristics: The conductor leads 23 vertically penetrate through and are connected to the electrodes 24 and the bonding pads 25, and the direct current resistance is less than 0.1Ω. The lead diameter of each of the conductor leads 23 ranges from 0.1 mm to 1 mm. The solid parts of the conductor leads 23 account for more than 50% of the volume of the through holes 212. That is, the conductor leads 23 may be hollow or solid conductors, but the lowest filling rate is 50%. Specifically, the interior of each of the conductor leads 23 may be of a compact structure, as shown in FIG. 12. The interior of each of the conductor leads 23 may also have pores 235, and the pores 235 are bubbles naturally formed in a process of screen-printing and filling the paste form metal of the metal or the alloy material into the through holes 212 to form the conductor leads 23. The conductor leads 23 and the substrate 21 of the porous ceramic structure are matched and co-sintered to form an integral structure, the formed conductor leads 23 and the through holes 212 of the substrate 21 are in a co-sintered embedding state, dismounting or falling cannot occur after the co-sintering and shaping, and the reliability of the conductor leads 23 is greatly improved. This application is different from a structure that the leads are arranged outside the substrate 21 or partially arranged inside the substrate 21 and partially arranged outside the substrate 21 in the prior art, and the problem that the conductor leads 23 may easily break off or be damaged through being pulled in the production and assembly process is solved.

The pores 235 may be through holes or blind holes, as shown in FIG. 13, the pores 235 may be in irregular pore shapes in the conductor leads 23. As shown in FIG. 14, the pores 235 may exist on the surface of the conductor leads 23, may exist inside the conductor leads 23, and are bubbles naturally formed by screen-printing and filling the paste form metal of the metal or alloy material into the through holes 212 to be sintered, their shapes and sizes may be in any states, and they are not limited in this application.

As shown in FIG. 15, the substrate 21 is of a porous structure, so that the side walls of the through holes 212 formed inside the substrate are in an uneven state. In an embodiment, the conductor leads 23 have protrusions 236 on the side walls, the through holes 212 have depressions 2121 on the side walls, and the protrusions 236 are embedded in the depressions 2121, so that the edges of the conductor leads 23 combined with the substrate 21 are uneven rough edges, the combination is firmer, the conductor leads 23 are prevented from falling off from the substrate 21, and the stability is high. In other embodiments, the side walls of the conductor leads 23 and the side walls of the through holes 212 may also be in a smooth state, and it is not limited in this application.

In this embodiment, as shown in FIG. 2, FIG. 3 and FIG. 11, an ejector pin 26 in direct contact with the bonding pad 25 is arranged at the bottom of each bonding pad 25, and is configured to conduct the heating element 22 and the power supply assembly 2. During working, the ejector pin 26 has a longitudinal stress direction, i.e., the direction from the first surface 213 to the second surface 214. When the ejector pin 26 exerts acting force, a mutual embedding structure of the substrate 21 and the conductor leads 23 may achieve a limiting effect, the conduction contact stability between the substrate and the conductor leads is enhanced, meanwhile, the mechanical property is excellent, the conductor leads 23 are prevented from falling off from the substrate 21, and the conduction is more stable.

The atomization core disclosed by this application includes a substrate, a heating element and conductor leads. The substrate has an atomization surface and through holes extending to the atomization surface. The substrate is configured to guide an aerosol-forming material to the atomization surface. The heating element is arranged on the atomization surface and is configured to heat and atomize the aerosol-forming material to form aerosols. The conductor leads are arranged in the through holes and fixed to the substrate to form an integral structure. A first end of each of the conductor leads is electrically connected to the heating element, and a second end of each of the conductor leads is configured to connect a power supply assembly. In this application, the conductor leads are arranged in the substrate to form an integral structure with the substrate, so that the connection direction of the heating element and a power supply is changed, the leads are prevented from shielding the atomization surface, the problems that the contact stability between the conductor leads and the substrate of the ceramic atomization core is poor, and damage may easily occur are solved, and the atomization conversion efficiency is improved to a greatest extent.

The foregoing descriptions are merely implementations of this application, and the patent scope of this application is not limited thereto. All equivalent structure or equivalent process changes made according to the content of this specification and accompanying drawings in this application or by directly or indirectly applying this application in other related technical fields shall fall within the protection scope of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An atomization core, comprising:
a substrate having an atomization surface and through holes extending to the atomization surface, the substrate being configured to guide an aerosol-forming material to the atomization surface;
a heating element arranged on the atomization surface and configured to heat and atomize the aerosol-forming material to form aerosols; and
conductor leads arranged in the through holes and fixed to the substrate to form an integral structure, a first end of each conductor lead of the conductor leads being electrically connected to the heating element, and a second end of each conductor lead of the conductor leads being configured to connect a power supply assembly.

2. The atomization core of claim 1, wherein the conductor leads have protrusions on side walls thereof, the through holes having depressions on the side walls, and the protrusions being embedded in the depressions.

3. The atomization core of claim 1, wherein the conductor leads comprise solid conductors, or the conductor leads have pores.

4. The atomization core of claim 1, wherein solid parts of the conductor leads comprise more than 50% of a volume of the through holes.

5. The atomization core of claim 1, wherein the substrate has a first surface and a second surface in opposite arrangement,
wherein the first surface comprises the atomization surface, and
wherein the through holes extend from the first surface to the second surface.

6. The atomization core of claim 5, wherein the through holes comprise straight-through holes perpendicular to the first surface.

7. The atomization core of claim 5, further comprising:
electrodes arranged on the first surface and electrically connected to the heating element; and
bonding pads arranged on the second surface and configured to be connected to the power supply assembly,
wherein the first end of each conductor lead is electrically connected to the electrode, and the second end of each conductor lead is electrically connected to the bonding pad.

8. The atomization core of claim 1, wherein a lead diameter of each conductor lead ranges from 0.1 mm to 1 mm, and/or
wherein a material of the conductor leads comprises at least one of Ag, Cu, and Au.

9. The atomization core of claim 1, wherein the substrate comprises a porous substrate, and a porosity of the substrate ranges from 30% to 80%, and/or
wherein a pore diameter of the pores of the substrate ranges from 10 um to 200 um.

10. The atomization core of claim 1, wherein the conductor leads are prepared by filling conductive paste into the through holes and then sintering.

11. The atomization core of claim 1, wherein the substrate has a first surface and a second surface in opposite arrangement,
wherein side surfaces are connected to the first surface and the second surface,
wherein the first surface comprises the atomization surface, and
wherein the through holes extend from the first surface to the side surfaces.

12. An atomizer, comprising:
a housing having an accommodating cavity; and
the atomization core of claim 1 arranged in the accommodating cavity, the atomization core being matched with the housing to form a liquid storage cavity, the atomization core being configured to heat and atomize an aerosol-forming material from the liquid storage cavity when powered on to form aerosols.

13. An electronic atomization apparatus, comprising:
the atomizer of claim 12; and
a power supply assembly electrically connected to the conductor leads of the atomizer and configured to supply power to the atomizer.
